(19) United States
(12) Patent Application Publication
(10) Pub. No.: US 2025/0339461 A1
(43) Pub. Date: Nov. 6, 2025

(54) METHODS OF TREATING MIGRAINE HEADACHE OR TENSION HEADACHE WITH HYALURONIC ACID

(71) Applicant: David Donaldson Brown, Garden Grove, CA (US)

(72) Inventor: David Donaldson Brown, Garden Grove, CA (US)

(21) Appl. No.: 19/270,124

(22) Filed: Jul. 15, 2025

Related U.S. Application Data
(63) Continuation of application No. PCT/US2025/011119, filed on Jan. 10, 2025.
(60) Provisional application No. 63/630,165, filed on Jan. 10, 2024.

(51) Int. Cl. A61K 31/728; A61K 9/00; A61P 25/00; A61P 25/06

(52) U.S. Cl. CPC A61K 31/728; A61K 9/0019; A61P 25/00; A61P 25/06

(57) ABSTRACT

The present invention provides methods of treating migraine headache in a subject, comprising administering to the subject, by one or more injections, a composition comprising hyaluronic acid (HA) and a pharmaceutically acceptable carrier. The injections are made into, or immediately adjacent to, soft tissue attachments to the subject's posterior skull. Also provided are methods of treating tension headache.

### CROSS-REFERENCE TO RELATED APPLICATIONS

This application is a continuation of International Application No. PCT/US2025/011119, filed on Jan. 10, 2025, which claims priority to U.S. Provisional Application No. 63/630,165, filed Jan. 10, 2024, the contents of each of which are herein incorporated by reference in their entirety.

### FIELD

This invention relates to the therapeutic treatment of headache. In particular, this invention relates to methods of treating or preventing migraine headache and/or tension headache with hyaluronic acid injections.

### BACKGROUND

Migraine headache affects a large fraction of the population, and diagnostic criteria have been elaborated by the International Headache Classification. Characteristic features of these headaches include throbbing pain, aura, nausea, light and sound sensitivity, as well as worsening with physical activity. A large variety of treatments for migraine headache are available, both for prophylaxis as well as acute pain relief/rescue, and many of these are quite effective. However, none of the existing treatments are uniformly effective at preventing or terminating migraine headaches, and for individuals with highly frequent migraines, combinations of preventive agents may be necessary, and often still do not eliminate their headaches.

Tension type headache is a recognized form of headache by the International Headache Classification, for which formal diagnostic criteria have been elaborated. Chronic tension type headache is a more frequent and problematic subtype of tension headache, in which people have tension headaches at least 15 days per month for at least three months. In some patients with chronic tension type headache, the pain is located primarily in the occipital region. Current treatments for chronic tension headache include physical therapy, prophylaxis with medications such as amitriptyline and topiramate, and over the counter analgesics for acute pain relief/rescue. However, for prophylactic treatments the results are generally unsatisfactory, in addition to being expensive and time consuming (physical therapy) and often lead to problematic side effects from medications used.

Therefore, a need exists for new methods of treating migraine headache and/or tension headache that demonstrate therapeutic efficacy, with reduced potential for deleterious side effects.

### SUMMARY

In an aspect, the present invention provides methods of treating or preventing migraine headache in a subject. The methods comprise administering to the subject, by one or more injections, a composition comprising hyaluronic acid (HA) and a pharmaceutically acceptable carrier. The injections are made into, or immediately adjacent to, soft tissue attachments to the subject's posterior skull. The one or more injections may be made into the soft tissue attachments within about ½″ to about 1″ from the superior and/or inferior nuchal line of the skull. The methods may further comprise palpating the attachment sites of the semispinalis capitis and/or splenius capitis to the posterior skull and selecting areas of pain or tenderness on palpating for injection.

In another aspect, the present invention provides methods of treating tension headache in a subject, comprising administering to the subject, by one or more injections, a composition comprising hyaluronic acid (HA) and a pharmaceutically acceptable carrier. The injections are made into, or immediately adjacent to, soft tissue attachments to the subject's posterior skull. The one or more injections may be made into the soft tissue attachments within about ½″ to about 1″ from the superior and/or inferior nuchal line of the skull. The methods may further comprise palpating the attachment sites of the semispinalis capitis to the posterior skull and selecting areas of pain or tenderness on palpating for injection.

### BRIEF DESCRIPTION OF THE DRAWINGS

FIG. 1 is a drawing illustrating the attachment sites of the semispinalis capitis [104], rectus capitis posterior major [102], rectus capitis posterior minor [101], and obliquus capitis inferior [103] to the posterior skull. The semispinalis capitis attaches in a broad band just inferior to the superior nuchal line [100]. The other muscles are deep to the semispinalis capitis, which is cut away on the left side of this figure. Although these figures show the deeper muscles attaching just inferior to the superior nuchal line, most sources show these muscles attaching just inferior to the inferior nuchal line.

FIG. 2 is a drawing illustrating example sites of injection of hyaluronic acid for treating migraine. Injections are given along the insertion sites of the semispinalis capitis [104] and/or splenius capitis [201] to the posterior skull. Sites on the right side of a subject marked with an “X” show the sites that are palpated and optionally injected with hyaluronic acid. Three sites for palpation and optional hyaluronic acid injection are marked at each location. The trapezius muscle [200] is superficial to and attaches to the posterior skull immediately superior to the semispinalis capitis attachment, and the muscles cannot be reliably differentiated using palpation.

FIG. 3 is a drawing illustrating the example sites on the left and right side of a subject, along the insertion sites of the semispinalis capitis [104], for injection of hyaluronic acid for treating chronic tension type headache manifesting in the occipital region. Sites marked with an “X” are palpated and optionally injected. Two sites for palpation and optional hyaluronic acid injection are marked on each side.

### DETAILED DESCRIPTION

There is no generally recognized anatomical origin or origins of the pain of migraine headache or tension headache. There also is no generally recognized type of headache arising from the major muscles, tendons, or their attachments to the posterior skull.

Hyaluronic acid (HA) is a large, naturally occurring polymer that is a major constituent of synovial fluid, tendon, ligament and extracellular matrix and is involved in lubricating joint movements. As used herein, “hyaluronic acid” or “HA” encompasses, HA, its conjugate base (hyaluronate), and derivatives thereof. In addition to its lubricating properties, HA exhibits anti-inflammatory properties that are dependent on concentration and molecular weight.¹

HA is FDA-approved as an injection treatment for osteoarthritis of the knee, where the therapeutic effect has been believed to be mediated primarily by its lubricant properties. In addition, HA has been investigated extensively for its use in other arthritic joints. While HA has been used with some degree of success in several tendinous disorders, including supraspinatus tendinosis, lateral epicondylitis, and patellar tendinopathy, no evidence has demonstrated the efficacy of HA in treating any type of headache disorder.²

As demonstrated in the Examples, the present inventor discovered that that injections of HA into the soft tissue attachments to the posterior skull are effective in treating migraine headache and tension headache, including tension headache that manifests pain in the occipital region. Thus, the present disclosure provides methods of treating migraine headache and tension headache comprising administering one or more HA injections. Chronic migraine headache and chronic tension headache are examples of chronic headache disorder. As used herein, a “chronic headache disorder” refers to a condition characterized by recurrent or persistent headaches over a period of at least 3 months. The methods and compositions provided may be used to treat any suitable chronic headache disorder, including, without limitation, chronic migraine headache, chronic tension headache, chronic cluster headache, and cervicogenic headache.

### Migraine Headache

In an aspect, the present invention provides methods of treating migraine headache in a subject. A “subject” or “patient” refers to any organism in need of and/or subjected to a treatment, such as a farm animal, domestic animal, or human. In some embodiments, the subject is a human.

In an aspect, the methods comprise administering to the subject a composition comprising a therapeutically effective amount of HA. HA is a polymer of D-glucuronic acid and N-acetyl-D-glucosamine that occurs naturally at varied sizes, with molecular weights ranging from 5 to 20,000 kilodaltons (kDa). The present methods may use HA having any suitable molecular weight. In some embodiments, the composition comprises HA having a molecular weight of about 50, 75, 100, 200, 300, 400, 500, 600, 700, 800, 900, 1000, 5000, 10000, 15000, or 20000 kDa, or a molecular weight within a range bounded by any of the forgoing. In some embodiments, the HA has a molecular weight of at least 500 kDa. In some embodiments, the HA has a molecular weight of about 500 kDa to about 730 kDa, or between about 450 kDa and 800 kDa, or between 400 kDa and 1000 kDa. The methods may suitably use linear HA or cross-linked HA. Suitable polymer cross-linking agents are known in the art and include, without limitation, cross-linking proteins/peptides, 1,4-Butanediol diglycidyl ether, and divinyl sulfone.

In some embodiments, the composition comprises a pharmaceutically acceptable salt of HA. As used herein, a “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the composition comprises a HA sodium salt.

The HA may be present in the composition at any suitable concentration. In some embodiments, the HA is present in the composition at a concentration of about 1 mg/mL, 5 mg/mL, 10 mg/mL, 15 mg/mL, 20 mg/mL, or 25 mg/mL, or at a concentration within a range bounded by any of the foregoing. In some embodiments, the HA is present in the composition at a concentration of about 10 mg/mL. The concentration of HA in the composition may be between 9-11 mg/mL, between 8-12 mg/mL, or between 5-15 mg/mL.

In an aspect, the composition further comprises a pharmaceutically acceptable carrier. Pharmaceutically acceptable carriers are known in the art and include, but are not limited to, diluents (e.g., Tris-HCl, acetate, phosphate), preservatives (e.g., Thimerosal, benzyl alcohol, parabens), solubilizing agents (e.g., glycerol, polyethylene glycerol), emulsifiers, liposomes, and nanoparticles. Pharmaceutically acceptable carriers may be aqueous or non-aqueous solutions, suspensions, or emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions, or suspensions, including saline and buffered media. The composition may further comprise additives such as albumin or gelatin to prevent absorption to surfaces, detergents (e.g., Tween 20, Tween 80, Pluronic F68, bile acid salts), anti-oxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc.) or onto liposomes, microemulsions, micelles, unilamellar, bilamellar or multilamellar vesicles, erythrocyte ghosts, or spheroplasts. The compositions may also be formulated in lipophilic depots (e.g., fatty acids, waxes, oils) for controlled or sustained release.

In some embodiments, the pharmaceutically acceptable carrier comprises sodium chloride, monobasic sodium phosphate, dibasic sodium phosphate, and water. HA is FDA-approved and commercially available as an injection treatment for osteoarthritis of the knee in several brands of differing molecular weights (including, without limitation, Hyalgan®, Synvisc®, Monovisc®, Orthovisc®, Durolane®). Thus, in some embodiments, the methods comprise administering a commercially available formulation.

As used herein, the term “therapeutically effective amount” refers to an amount of HA sufficient to treat a disease or disorder in a subject receiving the composition. Treating migraine headache includes reducing or alleviating the duration and/or severity of one or more symptoms of a migraine headache, including, without limitation, pain, aura, nausea, photosensitivity, auditory sensitivity, and neurologic accompaniments such as paresthesia and weakness. Treating migraine headache also includes reducing the frequency of migraine headache occurrence in the subject. Methods for assessing migraine treatment are known in the art and include, without limitation, assessments based on pain severity (average and/or maximum) and pain duration. In some embodiments, pain severity is assessed using a visual analog numerical rating scale ranging from 0 (no pain) to 10 (worst pain possible). Average and maximum pain severity may be assessed on a daily, weekly, or monthly basis. In some embodiments, the patient's average pain severity and/or maximum pain severity during a migraine headache is reduced following administration of the HA composition. In some embodiments, the total number of hours per day during which the patient experiences pain is reduced following administration of the HA composition. In some embodiments, the number of pain-free days per week and/or month is increased following administration of the HA composition. In some embodiments, the frequency of migraine headache occurrence is reduced following administration of the HA composition. The frequency of migraine headache occurrence may be assessed on a daily, weekly, or monthly basis.

The HA composition may be administered to any subject experiencing migraine headache. Patients experiencing migraine headache may be identified using any suitable criteria known in the art. In some embodiments, the composition is administered to a subject experiencing migraine headache as defined in by the International Classification of Headache Disorders. In some embodiments, the composition is administered to a subject experiencing chronic migraine headache. As used herein, “chronic migraine headache” refers to a condition characterized by headaches occurring on an average of 15 or more days per month for greater than 3 months, with headaches having the features of migraine headache on at least 8 days per month.

The composition is administered by one or more injections. The composition may be administered by 1, 2, 3, 4, 5, 6, 7, 8, 9, 10, 15, 20, 25, or 30 injections, or by a number of injections within a range bounded by any of the foregoing. In some embodiments, the composition is administered by 2-12 injections. The injections may be made using any suitable needle, including, without limitation, a 30 gauge, ½″ needle, and may deliver any suitable amount of HA. In some embodiments, each injection delivers about 0.25, 0.5, 0.75, 1, 1.5, 2, 2.5, 3, 4, 5, 6, 7, 8, 9, or 10 mL of the composition, or a volume of the composition within a range bounded by any of the foregoing. In some embodiments, each injection delivers about 0.5 mL to 1 mL of the composition. In some embodiments, each injection delivers 1, 2, 3, 4, 5, 6, 7, 8, 9, 10, 20, 30, 40, or 50 mg of HA, or an amount of HA within a range bounded by any of the foregoing. In some embodiments, each injection delivers 5 mg or more of HA. In some embodiments, each injection delivers 5 mg to 10 mg of HA. The amount of HA delivered may be independently selected for each injection. In some embodiments, the HA is administered by microneedling, which may comprise at least 10, at least 50, at least 100, at least 500, or at least 1000 injections, or any number of injections within a range bounded by any of the forgoing, and which may deliver (a) a total volume of the composition of about 0.25, 0.5, 0.75, 1, 1.5, 2, 2.5, 3, 4, 5, 6, 7, 8, 9, or 10 mL, or a total volume of the composition within a range bounded by any of the foregoing, and/or (b) a total amount of HA of about 1, 2, 3, 4, 5, 6, 7, 8, 9, 10, 20, 30, 40, or 50 mg of HA, or a total amount of HA within a range bounded by any of the foregoing.

In an aspect, the injections are made into, or immediately adjacent to the soft tissue attachments to the subject's posterior skull. As used herein, “soft tissues” refer to supportive and/or connective body tissues, including, without limitation, muscles, tendons, ligaments, and aponeuroses. Soft tissue attachments to the posterior skull include, without limitation, the fibro-osseous attachment sites of the splenius capitis muscle, semispinalis capitis muscle, trapezius muscle, rectus capitis posterior major and minor muscles, and obliquus capitis superior muscle. As used herein, a “fibro-osseous attachment” or “fibro-osseous attachment site” refers to a point at which a tendon or ligament attaches to bone. In some embodiments, the injections are made into the medial (toward the midline of the body) and/or lateral (away from the midline of the body) aspects of the semispinalis capitis insertion and/or the medial and lateral aspects of the splenius capitis insertion (as determined by palpation). As used herein, “origin” or “tendinous origin” and “insertion” or “tendinous insertion” refer to the two anchoring points of a muscle (one at each end), with the tendon attached to a more fixed bone at the origin and to a more mobile bone at the insertion. The semispinalis capitis and splenius capitis are superficial to other muscles that attach to the posterior skull, and injection of the insertions of these deeper muscles to the posterior skull may occur when injecting the insertions of the semispinalis capitis or splenius capitis, and such injections are encompassed in this invention. Injections may likewise be made into or spread into the occipitalis muscle, which attaches along the superior margin of the superior nuchal line. In some embodiments, the injections are made directly into the tendinous insertions or fibro-osseous attachments of these muscles to the posterior skull. Injections may acceptably be made into soft tissue immediately adjacent to the bony attachments of tendon to the palpated locations, as long as the injected volume of hyaluronic acid is sufficient to spread to these attachments. Larger injection volumes and dosages may allow for injection at greater distances from these locations. Injections may also be made into the muscle belly of any muscle that attaches to the posterior skull. In some embodiments, the injections are made into the soft tissue attachments within ½″ to about 1″ of the superior nuchal line and/or inferior nuchal line of the subject's skull.

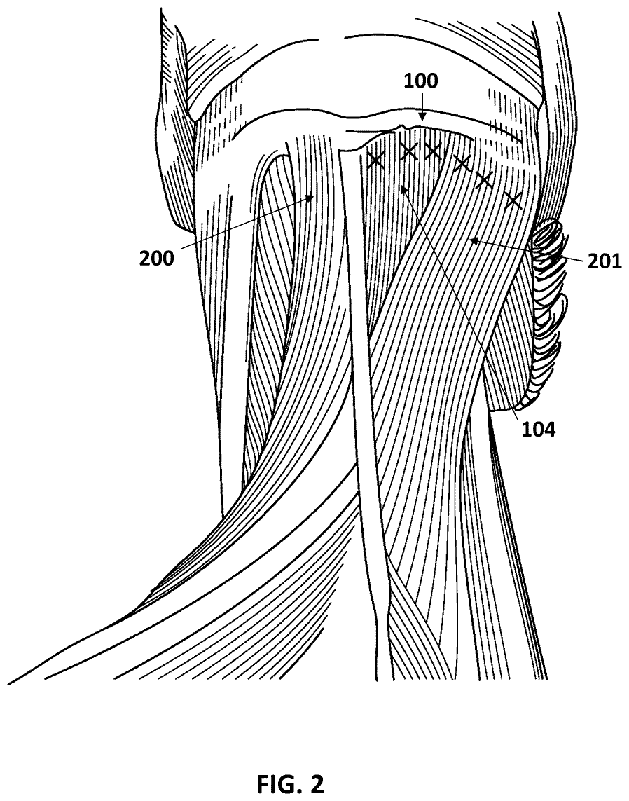

In some embodiments, the composition is administered by two or more injections, spaced at an interval. The two or more injections may be spaced at an interval of about ¼″, ½″, ¾″, 1″, 1¼″, 1½″, 1¾″, 2″, 3″, or 4″, or by an interval within a range bounded by any of the foregoing. In some embodiments, the two or more injections are spaced at an interval of about ½″ to about 1.″ In some embodiments, the methods further comprise identifying one or more injection sites for the one or more injections, comprising palpating the attachment sites of the semispinalis capitis and splenius capitis to the posterior skull and selecting areas of pain or tenderness on palpating for injection. As used herein, “palpating” or “palpate” refers to a manual examination designed to assess by touch/feel one or more of the size, shape, firmness, location, consistency, texture, and/or tenderness of a body part. The palpation may be used to detect focal tenderness at or near the medial and lateral aspects of each of the left and right semispinalis capitis insertion site, and/or the medial and lateral aspects of each splenius capitis insertion site. The focal tenderness may be identified when the subject vocalizes pain, winces, or moves their body in response to the pain elicited by firm palpation, and may reveal pain arising from muscle, tendinous attachments or underlying periosteum. Exemplary sites for palpation on the right side of a subject, along the insertion sites of the semispinalis capitis [104] and splenius capitis [201] are illustrated in FIG. 2, and are shown on the figure as marked with an “X”. Three sites for palpation are marked at each location. The sites injected may be limited to a single side of the subject or both the left and right side of the subject.

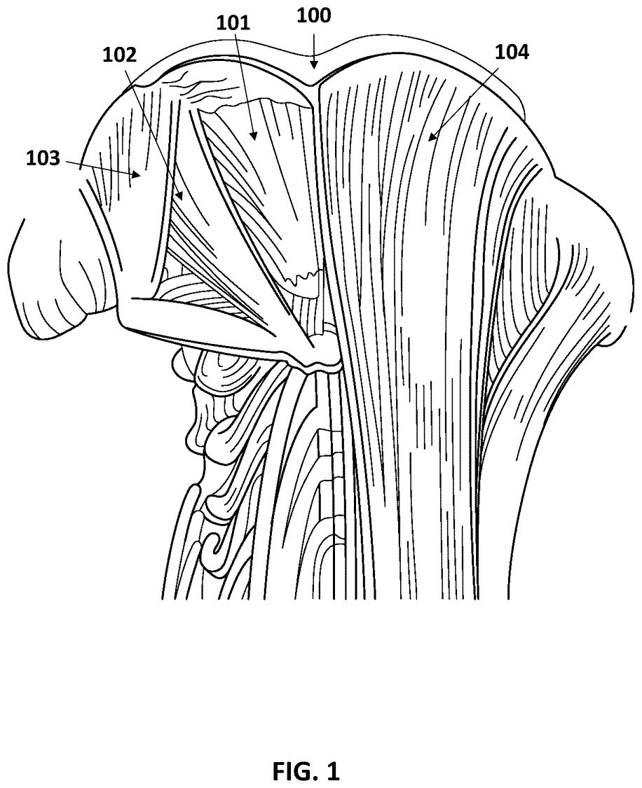

Any suitable method of palpation may be used to identify the one or more injection sites. For example, the medial semispinalis capitis insertion may be identified by palpating the posterior skull, beginning at its most posterior point, just immediately lateral to the midline on each side, and proceeding inferiorly until identifying the transition site from the bony prominence of the skull to soft tissue. The lateral aspect of the semispinalis capitis insertion may then be identified by palpating laterally from the medial semispinalis capitis insertion site by approximately ¾″ to 1″ until the interface between bone and soft tissue is identified again. In addition to lateral aspect of the semispinalis capitis, the injections may suitably be made into the soft tissue attachments of the trapezius (See, FIG. 2 at [200] and/or the deeper suboccipital muscles (e.g., the rectus capitis posterior major and minor (See, FIG. 1 at [102] and [101], respectively), as well as the obliquus capitis superior (See, FIG. 1 at [103])), which insert on the skull at a similar site.

The lateral aspect of the splenius capitis insertion may be identified by palpating the most inferior aspect of the lateral aspect of the mastoid process, and then proceeding to palpate posteriorly and medially along a horizontal line until soft tissue can be differentiated from bone. The medial aspect of the splenius capitis insertion may be identified by palpating along the skull at an approximately 30 degree angle from the horizontal, and approximately ¾″ to 1″ medially from the herein defined lateral aspect of the splenius capitis insertion, until a transition from bony skull to soft tissue is identified.

In some embodiments, the palpation may be performed as follows: (1) beginning at the posterior aspect of the mastoid process of one side, the interface between skull and soft tissue is identified, (2) proceeding along the posterior skull, the interface between skull and tissue is identified, and (3) injections are then made into the soft tissue at the bone/soft tissue interface along the posterior skull.

In some embodiments, areas of focal pain and/or tenderness identified by palpation are selected as injection sites, and the HA composition is injected into or immediately adjacent to the soft tissue attachments to these sites. The palpation may identify 1, 2, 3, 4, 5, 6, 7, 8, 9, 10, 15, 20, 25, or 30 injection sites, or a number of injection sites within a range bounded by any of the foregoing. In some embodiments, the one or more injections are made into 2-12 identified injection sites in a single treatment.

The composition may be administered using any suitable dosing scheme. For example, the composition may be administered daily, or once every 1, 2, 4, 6, 8, 10, 12, 24, or 36 weeks, or within a range bounded by any of the foregoing. In some embodiments, the composition is administered once per week. In other embodiments, the composition is administered once every 4 to 8 weeks, and the administration may be repeated every 4-8 weeks indefinitely, or may be administered on an as needed basis indefinitely. In some embodiments, the composition is administered during two or more treatment periods, each treatment period comprising administration once per week for 3 to 5 weeks, with the treatment periods separated by about 6 months.

### Tension Headache

In an aspect, the present invention also provides methods of treating tension headache in a subject. The methods comprise administering, by one or more injections, a composition comprising a therapeutically acceptable amount of HA and a pharmaceutically acceptable carrier as described above. The methods may suitably comprise administration of any disclosed composition, by any disclosed number of injections/injection sites, spaced at any disclosed interval, using any disclosed injection volume to deliver any disclosed quantity of HA, by any disclosed dosing scheme as described above.

In some embodiments, the one or more injections are made into the soft tissue attachments within about ½″ to about 1″ from the superior nuchal line and/or inferior nuchal line of the subject's skull. In some embodiments, the composition is administered by two or more injections along the subject's superior nuchal line and/or inferior nuchal line, and wherein the two or more injections are spaced at an interval of about ½″ to about 1″. In some embodiments, the composition is administered by 2-10 injections. In some embodiments, the HA has a molecular weight of at least 500 kDa. In some embodiments, the HA has a molecular weight of 500 kDa to 730 kDa. In some embodiments, the HA is present in the composition at a concentration of 5 mg/mL to 20 mg/mL. In some embodiments, the HA is present in the composition at a concentration of about 10 mg/mL. In some embodiments, each injection of the one or more injections delivers 5 mg or more of the HA. In some embodiments, each injection of the one or more injections delivers 5 mg to 10 mg of the HA. In some embodiments, the composition is administered once every 4 to 12 weeks. In some embodiments, the composition is administered during two or more treatment periods, each treatment period comprising administration once per week for 3 to 5 weeks, with the treatment periods separated by about 6 months. In some embodiments, the composition is administered once every 6 months.

Treating tension headache includes reducing or alleviating the duration or severity of one or more symptoms of a tension headache, including, without limitation, pain. Treating tension headache also includes reducing the frequency of tension headache occurrence in the subject. Methods for assessing tension headache treatment are known in the art and include, without limitation, assessments based on pain severity (average and/or maximum) and pain duration. In some embodiments, pain severity is assessed using a visual analog numerical rating scale ranging from 0 (no pain) to 10 (worst pain possible). Average and maximum pain severity may be assessed on a daily, weekly, or monthly basis. In some embodiments, the patient's average pain severity and/or maximum pain severity during a tension headache is reduced following administration of the HA composition. In some embodiments, the total number of hours per day during which the patient experiences pain is reduced following administration of the HA composition. In some embodiments, the number of pain-free days per week and/or month is increased following administration of the HA composition. In some embodiments, the frequency of tension headache occurrence is reduced following administration of the HA composition. The frequence of tension headache occurrence may be assessed on a daily, weekly, or monthly basis.

The HA composition may be administered to any subject experiencing tension headache. Patients experiencing tension headache may be identified using any suitable criteria known in the art. In some embodiments, the composition is administered to a subject experiencing tension headache as defined in by the International Classification of Headache Disorders. In some embodiments, the composition is administered to a subject experiencing chronic tension headache. As used herein, “chronic tension headache” refers to a condition characterized by tension headaches occurring on an average of 15 or more days per month for greater than 3 months. In some embodiments, the composition is administered to a subject experiencing tension type headache with peri-cranial tenderness. In some embodiments, the composition is administered to a subject experiencing chronic pain in the left, right or bilateral occipital regions, which pain may be present for greater than 1 hour, 2 hours, 4 hours, 6 hours, 12 hours, or 16 hours per day. In some embodiments, the subject previously met diagnostic criteria for chronic migraine headache.

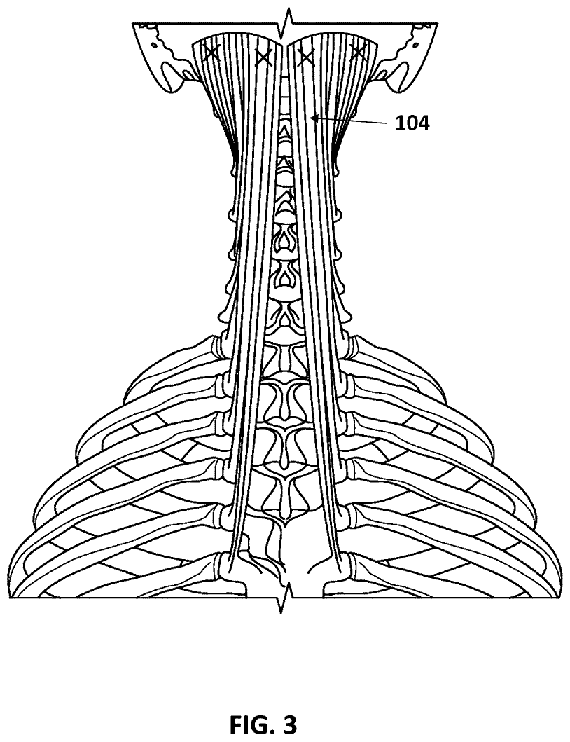

In some embodiments, the methods further comprise identifying one or more injection sites for the one or more injections, comprising palpating the attachment sites of the semispinalis capitis to the posterior skull and selecting areas of pain or tenderness on palpating for injection. The palpation may be used to detect focal tenderness at or near the left and/or right semispinalis capitis insertions on the skull, and this area of tenderness may overlap with or be adjacent to the insertions of the trapezius, rectus capitis posterior major, rectus capitis posterior minor, and obliquus capitis superior to the posterior skull. In some embodiments, palpable tenderness is identified by applying distinctly firm pressure to the posterior skull at the medial and lateral aspects of each of the left and right semispinalis capitis insertion sites. The focal tenderness may be identified when the subject vocalizes pain, winces, or move their body in response to the pain elicited by firm palpation, and may reveal pain arising from muscle, tendinous attachments, or periosteum, though distinction between these is not possible with palpation. The medial and lateral aspects of the semispinalis capitis may suitably be identified as described herein. In some embodiments, the injections are made into the medial and lateral aspects of the semispinalis capitis insertion. Exemplary sites for palpation on the left and right side of a subject, along the insertion sites of the semispinalis capitis [104] are illustrated in FIG. 3, and are shown on the figure as marked with an “X”. Two sites for palpation are marked on each of the left and right semispinalis capitis. The injections may also suitably be made into the soft tissue attachments of the trapezius and/or the deeper suboccipital muscles (e.g., the rectus capitis posterior major and minor (See, FIG. 1 at [102] and [101], respectively), as well as the obliquus capitis superior (See, FIG. 1 at [103])), which insert on the skull at a similar site. In some embodiments, the one or more injections are made into 2-10 identified injection sites in a single treatment.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument, and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXEMPLARY EMBODIMENTS

Embodiment 1. A method of treating headache in a subject, comprising administering to the subject a composition comprising (a) a therapeutically effective amount of hyaluronic acid and (b) a pharmaceutically acceptable carrier, wherein the composition is administered by one or more injections into, or immediately adjacent to, soft tissue attachments to the subject's posterior skull.

Embodiment 2. The method of embodiment 1, wherein the headache is a migraine headache.

Embodiment 3. The method of embodiment 2, further comprising identifying one or more injection sites for the one or more injections, wherein the identifying comprises palpating the attachment sites of the semispinalis capitis and/or splenius capitis to the posterior skull and selecting areas of pain or tenderness on palpating for injection.

Embodiment 4. The method of any one of embodiments 2-3, wherein the one or more injections are made into 2-12 injection sites.

Embodiment 5. The method of embodiment 1, wherein the headache is a tension headache.

Embodiment 6. The method of embodiment 5, further comprising identifying one or more injection sites for the one or more injections, wherein the identifying comprises palpating the attachment sites of the semispinalis capitis to the posterior skull and selecting areas of pain or tenderness on palpating for injection.

Embodiment 7. The method of any one of embodiments 5-6, wherein the one or more injections are made into 2-10 injection sites.

Embodiment 8. The method of any one of embodiments 1-7, wherein the one or more injections are made into the soft tissue attachments within about ½″ to about 1″ from the superior nuchal line and/or inferior nuchal line of the subject's skull.

Embodiment 9. The method of any one of embodiments 1-8, wherein the composition is administered by two or more injections along the subject's superior nuchal line and/or inferior nuchal line, and wherein the two or more injections are spaced at an interval of about ½″ to about 1″.

Embodiment 10. The method of any one of embodiments 1-9, wherein the hyaluronic acid has a molecular weight of at least 500 kDa.

Embodiment 11. The method of embodiment 10, wherein the hyaluronic acid has a molecular weight of 500 kDa to 730 kDa.

Embodiment 12. The method of any one of embodiments 1-11, wherein the hyaluronic acid is present in the composition at a concentration of 5 mg/mL to 20 mg/mL.

Embodiment 13. The method of embodiment 12, wherein the hyaluronic acid is present in the composition at a concentration of about 10 mg/mL.

Embodiment 14. The method of any one of embodiments 1-13, wherein each injection of the one or more injections delivers 5 mg or more of the hyaluronic acid.

Embodiment 15. The method of embodiment 14, wherein each injection of the one or more injections delivers 5 mg to 10 mg of the hyaluronic acid.

Embodiment 16. The method of any one of embodiments 1-15, wherein the composition is administered once every 4 to 12 weeks.

Embodiment 17. The method of any one of embodiments 1-15, wherein the composition is administered during two or more treatment periods, wherein (i) the composition is administered once per week for 3 to 5 weeks during each treatment period, and (ii) the treatment periods are separated by about 6 months.

Embodiment 18. The method of any one of embodiments 1-15, wherein the composition is administered once every 6 months.

Embodiment 19. The method of any one of embodiments 1-18, wherein the one or more injections are made into one or more sites as shown in FIGS. 2-3.

Embodiment 20. The method of any one of the previous embodiments, wherein the headache is a chronic headache.

### EXAMPLES

### Example 1 (Migraine)

A 61-year-old female patient with history of headaches for the past 4 to 5 years, reported constant pain in the back of her head and temple region, ranging from 5-8 out of 10 in severity on a visual analog numerical rating scale ranging. The more severe headaches occurred 5 to 6 days/week, worsened with physical activity, and were accompanied by nausea and sound sensitivity. Examination by palpation as described herein revealed distinct superior nuchal line tenderness. MRIs had previously been taken of the patient's cervical spine, but none of her brain. The patient reported no benefit from previous treatment with baclofen, gabapentin, propranolol, or ibuprofen, but ondansetron had been helpful for nausea. The patient was diagnosed with chronic migraine and prescribed riboflavin 200 mg bid, magnesium oxide 500 mg tid, and coenzyme Q10 100 mg bid, but after 4-6 weeks this did not affect the frequency of her headaches, which were occurring daily. She tried rizatriptan for rescue (pain relief) but had a severe elevation in her blood pressure. An MRI of her brain revealed a solitary microhemorrhage, most likely due to trauma or hypertension. The patient reported no history of head trauma, but did have significant hypertension for many years, though the chronic hypertension was not currently present, nor did the patient require blood pressure medication. The patient was administered hyaluronic acid injections (Hyalgan 20 mg/2 ml; Lot #: H12020) along the superior nuchal line from the insertion of splenius capitus on left to insertion of splenius capitis on right, including the insertions of the semispinalis capitis bilaterally. The hyaluronic acid was administered at a total of 12 injection sites, spaced approximately ½″ apart, with 0.5 mL administered at each injection site.

Approximately 6 weeks later, the patient reported that migraine headache frequency had decreased to occurring 2 to 3 days a week. She also reported substantially fewer of the more excruciating headaches, or headaches severe enough that she had to lie down and avoid lights, but that she was still having throbbing headaches 2 to 3 days/week. She was scheduled for Botox to complement the hyaluronic acid injections.

### Example 2 (Migraine)

A 45-year-old female patient reported a history of headaches starting she was approximately 2 years old, with migraine type headaches beginning around 10 years old. At present, the migraine headaches occurred essentially every day, lasting essentially all day. The migraine headache symptoms included throbbing, nausea, light and sound sensitivity worsening with physical activity, and some tearing and nasal congestion. The patient did not report any brief lancinating pains. Examination by palpation as described herein revealed distinct superior nuchal line tenderness. Previously conducted brain imaging was unremarkable. She had tried multiple prophylactic agents for these migraines without benefit, including amitriptyline, Ajovy, baclofen, carbamazepine, coenzyme Q10, Emgality, gabapentin, magnesium, Nurtec, prednisone, pregabalin, propranolol, tizanidine, topiramate, valproic acid, and zonisamide. None of these had been helpful, and several had caused deleterious side effects. The patient reported that Botox had been helpful, typically reducing the frequency in half (occurring approximately half the days of the month), but that she not been injected in over 6 months. For rescue, the patient reported that Excedrin Migraine caused side effects, Nurtec was not helpful, sumatriptan tablet was not helpful, Fioricet was partially helpful, sumatriptan injection was partially helpful, Ubrelvy was not helpful, and tramadol was partially helpful. She was intolerant of metoclopramide, but Zofran was helpful for nausea. The patient was diagnosed with chronic migraine and administered hyaluronic acid (Hyalgan 20 mg/2 mL; Lot #: G37480 and H12020) as follows: 0.5 mL was injected approximately every half inch along the superior nuchal line, beginning at the insertion of the left splenius capitis and traversing through the semispinalis capitis insertions bilaterally and to the right splenius capitis insertion.

Approximately 4 weeks later, the patient reported that the headaches were significantly improved for approximately 2 weeks after the hyaluronic acid injections, with less intense symptoms, requiring less rescue medicine. During this time, the patient still experienced frequent migraines, though they were not as intense/severe as those prior to the hyaluronic acid injections.

Headache intensity/severity, however, had returned to its normal baseline after four weeks. She was consequently administered Botox, though expressed interest in repeating the hyaluronic acid injections.

### Example 3 (Migraine)

A 52-year-old female patient reported a history of headaches since she was 9 years old, with current severe headaches occurring approximately 5 to 7 days/week, despite her current medication regimen. Headache symptoms included throbbing, light and sound sensitivity, and intensity was markedly worse with physical activity. The headaches lasted 6 to 8 hours and were primarily occipital. Examination by palpation as described herein revealed superior nuchal line tenderness. An MRI of her brain in 2014 showed a few punctate white matter hyperintensities. For migraine prophylaxis she had tried Botox several times, which initially gave good results, but the effectiveness gradually wore off over repeated injections. She was taking topiramate 200 mg nightly which helped some, though still frequent severe migraine headaches 5 to 7 days a week. For rescue, eletriptan was consistently effective, while rizatriptan was no longer effective. She was prescribed Emgality for chronic migraine and stated headaches were significantly improved, and that Nurtec was helpful for rescue. At this time she estimated she was having migraines 2 to 3 days/week, occasionally 4 days/week, and mild headaches on the other days. She received a total of 12 hyaluronic acid (Hyalgan 20 mg/2 ml; Lot #: H12020) injections as follows:
Left superior nuchal line: 6 injections (0.5 mL each) along the insertions of splenius capitis, semispinalis capitis, and trapezius, approximately 0.5″ apart
Right superior nuchal line: 6 injections (0.5 mL each) along the insertions of splenius capitis, semispinalis capitis, and trapezius, approximately 0.5″ apart

Two weeks after the hyaluronic acid injection she stated that she had not had any migraines since the injection, and has not had anything more than mild head pain, not qualifying as headache in her opinion, and not requiring any medication. Four weeks after the injection she stated the migraine headaches remained under excellent control, and stated she has only had 1 migraine since the injection.

### Example 4 (Migraine)

A 46-year-old female reported a history of headaches for the last 32 years, beginning at the age of 14, after a traumatic head injury. The patient reported multiple previous traumatic head injuries. At present, the patient reported constant severe headaches, lasting all day every day, with nausea, light and sound sensitivity, and worsening with physical activity. Examination by palpation as described herein revealed severe superior nuchal line tenderness. A previous MRI showed white matter disease, and a lumbar puncture with normal opening pressure. For migraine prophylaxis, she had tried amlodipine, baclofen, gabapentin, losartan, topiramate, and others with no benefit. She stated that Botox reduces the intensity of the migraine headaches, but did not impact the frequency or duration. She had started Emgality a few months prior to her visit, and had not noticed any obvious benefit. For rescue she had tried butorphanol, diclofenac, Excedrin Migraine, Migranal nasal spray, Reyvow, sumatriptan tablet, sumatriptan injection, tramadol, and zolmitriptan nasal spray without benefit. Ondansetron was at times helpful for nausea. She received a local anesthetic injection with bupivacaine along her superior nuchal line and pain was substantially improved for two days. She then received several rounds of Emgality, which reduced the pain somewhat, but had not reduced the frequency or duration, and had caused unwanted side effects. She was subsequently treated with Botox for her migraines, and although initially helpful at reducing the frequency, after several rounds of Botox, the effect was not as noticeable, and the patient reported daily severe migraine headaches, which had been occurring for the past 2 months, despite the last Botox injection two months prior. She was diagnosed with chronic migraine and enthesopathy, and administered 12 total hyaluronic acid (Hyalgan 20 mg/ml; Lot #: H12040) injections as follows:
Left superior nuchal line from, lateral to medial: 6 injections (0.5 mL each)
Right superior nuchal line, from lateral to medial: 6 injections (0.5 mL each)

After receiving the hyaluronic acid injection, the patient reported that migraine pain gradually improved over several hours, and the migraines, although daily, were less intense for a couple of weeks after the injection, averaging 5-7/10 in severity, compared to 8/10 in severity prior to HLA, the latter pain level typically being on the border of when she will go to the hospital. However, the effect of the HLA injections wore off after the end of approximately two weeks.

### Example 5 (Migraine)

A 32-year-old female reported headaches for the past 25 years, with no significant change in headache frequency over the past several years. She had mild headaches daily, at the base of the skull top and neck, present all day most days, with migraine headaches occurring approximately 2 to 3 days/week, and lasting several hours to 8 hours. Migraine symptoms included throbbing, nausea, light and sound sensitivity, and worsening with physical activity. For prophylaxis she had tried propranolol (caused dizziness), topiramate (could not tolerate the side effects), tizanidine (partially helpful), magnesium (partially helpful), and coenzyme Q10 (no benefit). Rizatriptan worked for rescue approximately 20% of the time. Botox was initially helpful, migraines went from every other day, approximately 15/month, to approximately once per week, approximately 4 to 5/month. However, the subject became pregnant and migraine headaches became more frequent. She was diagnosed with chronic migraine and enthesopathy, and administered 12 total hyaluronic acid (Hyalgan 20 mg/ml; Lot #: H12040) injections as follows:
Left superior nuchal line from, lateral to medial: 6 injections (0.5 mL each)
Right superior nuchal line, from lateral to medial: 6 injections (0.5 mL each)

After the hyaluronic acid injection, the migraine headaches were better for a few days, but the effect wore off, without a significant change in the frequency of her migraines over the course of the ensuing month.

### Example 6 (Tension Headache)

A 49 year old female patient reported constant dull pain in her left occipital region, present all day every day. This was not associated with any throbbing, nausea, light or sound sensitivity. She had comorbid neuropathic facial pain, treated effectively by pregabalin, and had focal tenderness at her left semispinalis capitis insertion. She received dexamethasone injections, which helped for several months each time she received them, but treatment with dexamethasone was ceased due to concerns regarding the effects of repeat steroid injections. The patient was administered hyaluronic acid (Hyalgan 10 mg/ml; Batch: G35650) injections as follows: after confirmation of focal tenderness to palpation performed as described herein, 1 mL of hyaluronic acid was injected into the medial insertion point of the left semispinalis capitis insertion, and 1 mL hyaluronic acid was injected into the lateral insertion of the left semispinalis capitis insertion point.

At follow up 8 weeks later, she stated the pain in her left occipital region was resolved for six weeks after the injections, but had begun to recur. She was treated again with hyaluronic acid (Hyalgan 10 mg/ml; Batch: G35650) injection, using the injection scheme described above.

When seen 8 weeks later, she stated the pain in left occipital region was well controlled. At the next appointment 8 weeks later, the left occipital region pain had recurred, and she was injected again with hyaluronic acid (Hyalgan 10 mg/ml; Batch: G37030), using the injection scheme described above. After this injection, the dull occipital pain remained well controlled for at least 8 weeks.

### Example 7 (Tension Headache)

A 47 year old female reported chronic headaches, occurring 6-7 days a week, reaching a severity 7 out of 10. The headaches would begin in the late morning or early afternoon, lasting rest of the day, and the symptoms included throbbing, with light and sound sensitivity. Examination revealed superior nuchal line tenderness. She was prescribed Emgality, and three weeks later reported that the headaches with throbbing, light and sound sensitivity had decreased to around once or twice per week, and that Nurtec was beneficial as a rescue agent. However, despite the reduction in migraine frequency, she continued to have constant moderate pain in her occipital region, present all day every day, with an average pain severity of 6/10 and maximum severity of 7 to 8 out of 10. Palpation as described herein revealed tenderness in her bilateral semispinalis capitis insertions. Hyaluronic acid (Hyalgan 20 mg/2 ml; Lot #: H12020) was injected as follows:
Medial and lateral aspect of left semispinalis capitis insertion: 2 injections of 0.5 mL (total of 1.0 mL)
Medial and lateral aspect of right semispinalis capitis insertion: 2 injections of 0.5 mL (total of 1.0 mL)

When evaluated one month later, the average pain level in her occipital region had decreased to 4/10 on average, with a maximum severity of 5-6/10. The patient stated that she felt significantly improved, despite the mild persistent pain.

### Example 8 (Tension Headache)

A 19-year-old female patient complained of headaches occurring since a motor vehicle accident approximately 2 to 3 months prior to the appointment. She reported moderately severe headaches in the occipital region, more prominently on the left. The headaches were present in the morning, lasting until approximately 1 PM each day, with pain averaging between 6 and 8 on a visual analog scale. This daily pain was not associated with any nausea, throbbing, light or sound sensitivity. She had more severe headaches approximately 2-3 times per month, which were also occipital and left-sided, with associated throbbing, nausea, and worsening with physical activity, as well as dizziness, with symptoms lasting all day or for a couple days. An MRI of her brain was conducted, which was unremarkable. Palpation as described herein revealed superior nuchal line tenderness, which was more notable in the semispinalis capitis region on the left than the right. The patient was diagnosed with chronic tension type headache with pericranial tenderness-occipital variant and semispinalis capitis enthesopathy. Hyaluronic acid injections (Hyalgan 20 mg/2 ml; Lot #: H12020) were administered as follows:
1 injection (0.5 mL each) at each of the medial and lateral aspect of left semispinalis capitis insertion;
1 injection (0.5 mL each) at each of medial and lateral aspect of right semispinalis capitis insertion.

Five days after the hyaluronic acid injection, the patient reported that the daily occipital pain was significantly improved, becoming more moderate, and averaging between 5-6 out of 10 on a visual analog scale.

### Example 9 (Tension Headache)

A 58 year old female with an underlying autoimmune myopathy, for which she was taking chronic prednisone, IVIG (for several years), and azathioprine, complained of constant occipital pain, present all day every day, that could become moderately severe, up to 8 out of 10. The pain was primarily occipital, but with no associated throbbing, no nausea, no light or sound sensitivity, and headaches may resolve with ibuprofen. On exam she had bilateral semispinalis capitis insertion tenderness. She received hyaluronic acid injections (Hyalgan 20 mg/ml; Lot #: H12050) as follows:
1 injection (0.5 mL each) at each of the medial and lateral aspect of left semispinalis capitis insertion;
1 injection (0.5 mL each) at each of medial and lateral aspect of right semispinalis capitis insertion.

Three weeks after the injections, the patient stated that the headaches were much better, with a maximum severity of 4-5/10, and only occurring 2 to 3 days week, with the other days free of headache.

### REFERENCES

1. Neumann et al., High molecular weight hyaluronic acid inhibits advanced glycation endproduct-induced NF-kB activation and cytokine expression, FEBS Letters 1999; 453:283-87.
2. Agostini F et al., Effects of hyaluronic acid injections on pain and functioning in patients affected by tendinopathies: A narrative review, Journal of Back and Musculoskeletal Rehabilitation, 2022; 35:949-961.

What is claimed:

1. A method of treating headache in a subject, comprising administering to the subject by one or more injections a composition comprising (a) a therapeutically effective amount of hyaluronic acid and (b) a pharmaceutically acceptable carrier, wherein the one or more injections are made into, or immediately adjacent to, muscle bellies, or soft tissue attachments thereof, of one or more muscles that attach to the subject's posterior skull.

2. The method of claim 1, wherein the headache is a migraine headache.

3. The method of claim 2, wherein the one or more injections are made into the soft tissue attachments within about ½″ to about 1″ from the superior nuchal line or inferior nuchal line of the subject's skull.

4. The method of claim 2, wherein the composition is administered by two or more injections along the subject's superior nuchal line and/or inferior nuchal line, and wherein the two or more injections are spaced at an interval of about ½″ to about 1″.

5. The method of claim 2, further comprising identifying one or more injection sites for the one or more injections, wherein the identifying comprises palpating the attachment sites of a semispinalis capitis, trapezius, and/or splenius capitis to the posterior skull and selecting areas of pain or tenderness on palpating for injection.

6. The method of claim 5, wherein the one or more injections are made into 2-12 injection sites.

7. The method of claim 2, wherein:
(i) the hyaluronic acid has a molecular weight of at least 500 kDa;
(ii) the hyaluronic acid is present in the composition at a concentration of 1 mg/mL to 25 mg/mL; and/or
(iii) each injection of the one or more injections delivers 1 mg or more of the hyaluronic acid.

8. The method of claim 2, wherein:
(i) the hyaluronic acid has a molecular weight of 500 kDa to 730 kDa;
(ii) the hyaluronic acid is present in the composition at a concentration of about 10 mg/mL; and/or
(iii) each injection of the one or more injections delivers 5 mg to 10 mg of the hyaluronic acid.

9. The method of claim 2, wherein the composition is administered:
(i) once every 4 to 12 weeks;
(ii) during two or more treatment periods, wherein the composition is administered once per week for 3 to 5 weeks during each treatment period, and wherein the treatment periods are separated by about 6 months; or
(iii) once every 1 to 36 weeks.

10. The method of claim 2, wherein the one or more injections are made into one or more sites as shown in FIG. 2.

11. The method of claim 1, wherein the headache is a tension headache.

12. The method of claim 11, wherein the one or more injections are made into the soft tissue attachments within about ½″ to about 1″ from the superior nuchal line and/or inferior nuchal line of the subject's skull.

13. The method of claim 11, wherein the composition is administered by two or more injections along the subject's superior nuchal line and/or inferior nuchal line, and wherein the two or more injections are spaced at an interval of about ½″ to about 1″.

14. The method of claim 11, further comprising identifying one or more injection sites for the one or more injections, wherein the identifying comprises palpating the attachment sites of a semispinalis capitis and/or trapezius to the posterior skull and selecting areas of pain or tenderness on palpating for injection.

15. The method of claim 14, wherein the one or more injections are made into 2-10 injection sites.

16. The method of claim 11, wherein:
(i) the hyaluronic acid has a molecular weight of at least 500 kDa;
(ii) the hyaluronic acid is present in the composition at a concentration of 1 mg/mL to 25 mg/mL; and/or
(iii) each injection of the one or more injections delivers 1 mg or more of the hyaluronic acid.

17. The method of claim 11, wherein:
(i) the hyaluronic acid has a molecular weight of 500 kDa to 730 kDa;
(ii) the hyaluronic acid is present in the composition at a concentration of about 10 mg/mL; and/or
(iii) each injection of the one or more injections delivers 5 mg to 10 mg of the hyaluronic acid.

18. The method of claim 11, wherein the composition is administered:
(i) once every 4 to 12 weeks;
(ii) during two or more treatment periods, wherein the composition is administered once per week for 3 to 5 weeks during each treatment period, and wherein the treatment periods are separated by about 6 months; or
(iii) once every 1-36 weeks.

19. The method of claim 11, wherein the one or more injections are made into one or more sites as shown in FIG. 3.

20. The method of claim 1, wherein the headache is a chronic headache.
